SEQUENCE LISTING (32 sequences)

SEQ ID NO: 1 - DNA, 1904 bp, Artificial Sequence
  mouse Bestrophin 1 gene (NM_011913)
gacccaagcccacctactgctgcccagtgccaagccatgactatcacctacacaaacaaagtagccaatgcccgcctcggttcgttctcgtccctcctcctgtgctggcgaggcagcatctacaagctgctgtatggagaattccttgtcttcatattcctctactattccatccgtggactctacagaatggttctctcgagtgatcagcagctgttgtttgagaagctggctctgtactgcgacagctacatccagctcatccctatatccttcgttctgggtttctatgttacattggtggtgagccgctggtggagccagtacgagaacttgccgtggcccgaccgcctcatgatccaggtgtctagcttcgtggagggcaaggatgaggaaggccgtttgctgcggcgcacgctcatccgctacgccatcctgggccaagtgctcatcctgcgcagcatcagcacctcggtctacaagcgctttcccactcttcaccacctggtgctagcaggttttatgacccatggggaacataagcagttgcagaagttgggcctaccacacaacacattctgggtgccctgggtgtggtttgccaacttgtcaatgaaggcctatcttggaggtcgaatccgggacaccgtcctgctccagagcctgatgaatgaggtgtgtactttgcgtactcagtgtggacagctgtatgcctacgactggataagtatcccattggtgtacacacaggtggtgacagtggcagtatacagctttttccttgcatgcttgatcgggaagcagtttctgaacccaaacaaggactacccaggccatgagatggatctggttgtgcctgtcttcacaatcctgcaattcttattctacatgggctggctgaaggtggcagaacagctcatcaaccccttcggggaggacgatgatgattttgagactaactggatcattgacagaaacctgcaggtgtccctgttgtccgtggatgggatgcaccagaacttgcctcccatggaacgtgacatgtactggaacgaggcagcgcctcagccgccctacacagctgcttctgccaggtctcgccggcattccttcatgggctccaccttcaacatcagcctaaagaaagaagacttagagctttggtcaaaagaggaggctgacacggataagaaagagagtggctatagcagcaccataggctgcttcttaggactgcaacccaaaaactaccatcttcccttgaaagacttaaagaccaaactattgtgttctaagaaccccctcctcgaaggccagtgtaaggatgccaaccagaaaaaccagaaagatgtctggaaatttaagggtctggacttcttgaaatgtgttccaaggtttaagaggagaggctcccattgtggcccacaggcacccagcagccaccctactgagcagtcagcaccctccagttcagacacaggtgatgggccttccacagattaccaagaaatctgtcacatgaaaaagaaaactgtggagtttaacttgaacattccagagagccccacagaacatcttcaacagcgccgtttggaccagatgtcaaccaatatacaggctctaatgaaggagcatgcagagtcctatccctacagggatgaagctggcaccaaacctgttctctatgagtgatgcctcacagcctggccctgacttgcaaggatgcccagcagggcactgacccagtcaaaggcacacaagcagcgacacccaggagtgtgttcccacgacagtctagcatgtaactcagaaccaagagtacttaatagtcctgcctgaaaacacctgtattttacgatctttcccaaactaaggagtttaataaacgtgaatattcttttagg

SEQ ID NO: 2 - DNA, 2673 bp, Artificial Sequence
  Human bestrophin 1 gene (NM_004183)
cagggagtcccaccagcctagtcgccagaccttctgtgggatcatcggacccacctggaaccccacctgacccaagcccacctgctgcagcccactgcctggccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaacctgcttcctaatggggatgcttcgccagccaggtcctcacctgtgtgtacaccagcaggacactgatccagtcacagccatacagctgtccacactgaagaacatgtcctacaacagcctgaatcaaatggttagcttaatagataaaaatcccagactacttcagcctttaatgccttttattcataaaaactgtgaaagctagactgaaccattggaaacatttaactcagactctggattcagagtcgggaacccttagttctatctgaatccaagacagccacaccttagtatactgcccaaactaatgagtttaataaatacaaatactcgtttctttttgattagtgtgattagaactgaacaacggcacttaaggaatctggaagatagcctggatagatttctgattcatcccaagacctcaaagacaacacctgggtaccaaatttctttatttgaaggaatggtacaaatcaaagaacttaagtggatgttttggtacaacttatagaaaaggtaaaggaaaccccaacatgcatgcactgccttggtgaccagggaagtcaccccacggctatggggaaattagcccgaggcttagctttcattatcactgtctcccagggtgtgcttgtcaaagagatattccgccaagccagattcgggcgctcccatcttgcgcaagttggtcacgtggtcacccaattctttgatggctttcacctgctcattcaggtaatgtgtctcaatgaagtcacacaactgcaaaacaatggggaagacagttagtgggcagctttccca

SEQ ID NO: 3 - DNA, 60 bp, Artificial Sequence
  cDNA for shRNA (5′->3′) for Best1 gene
gatccccttgccaacttgtcaatgaattcaagagattcattgacaagttggcaattttta

SEQ ID NO: 4 - DNA, 60 bp, Artificial Sequence
  cDNA for shRNA (3′->5′) against Best1 gene
gggaacggttgaacagttacttaagttctctaagtaactgttcaaccgttaaaaattcga

SEQ ID NO: 5 - DNA, 20 bp, Artificial Sequence
  mBest1-F primer
aggacgatgatgattttgag

SEQ ID NO: 6 - DNA, 20 bp, Artificial Sequence
  mBest1-R primer
ctttctggtttttctggttg

SEQ ID NO: 7 - DNA, 20 bp, Artificial Sequence
  mBest2-F primer
tcgtctacacccaggtagtc

SEQ ID NO: 8 - DNA, 20 bp, Artificial Sequence
  mBest2-R primer
gaaagttggtctcaaagtcg

SEQ ID NO: 9 - DNA, 20 bp, Artificial Sequence
  mBest4-F primer
aaaggctacgtaggacatga

SEQ ID NO: 10 - DNA, 20 bp, Artificial Sequence
  mBest4-R primer
gaaaggacggtatgcagtag

SEQ ID NO: 11 - DNA, 20 bp, Artificial Sequence
  mCLCA1,2,4-F primer
ttcaagatccaaaaggaaaa

SEQ ID NO: 12 - DNA, 20 bp, Artificial Sequence
  mCLCA1,2,4-R primer
gctcagtctggttttgtttc

SEQ ID NO: 13 - DNA, 20 bp, Artificial Sequence
  mCLCA5-F primer
taagattccagggacagcta

SEQ ID NO: 14 - DNA, 20 bp, Artificial Sequence
  mCLCA5-R primer
aaaggaggaaaaatacctgg

SEQ ID NO: 15 - DNA, 20 bp, Artificial Sequence
  mTtyh1-F primer
agacacctatgtgctgaacc

SEQ ID NO: 16 - DNA, 20 bp, Artificial Sequence
  mTtyh1-R primer
agaaaagagcatcaggaaca

SEQ ID NO: 17 - DNA, 20 bp, Artificial Sequence
  mTtyh2-F primer
ccagcttctgctaaacaact

SEQ ID NO: 18 - DNA, 20 bp, Artificial Sequence
  mTtyh2-R primer
aatctctgtccctgttgatg

SEQ ID NO: 19 - DNA, 20 bp, Artificial Sequence
  mTtyh3-F primer
cagtactgagtggggacatt

SEQ ID NO: 20 - DNA, 20 bp, Artificial Sequence
  mTtyh3-R primer
ctgtgacaaaggagaagagg

SEQ ID NO: 21 - DNA, 20 bp, Artificial Sequence
  mBest1 forward outer primer
aggacgatgatgattttgag

SEQ ID NO: 22 - DNA, 20 bp, Artificial Sequence
  mBest1 forward inner primer
accttcaacatcagcctaaa

SEQ ID NO: 23 - DNA, 20 bp, Artificial Sequence
  mBest1 reverse common primer
ctttctggtttttctggttg

SEQ ID NO: 24 - DNA, 20 bp, Artificial Sequence
  NSE forward common primer
gctgcctctgagttttaccg

SEQ ID NO: 25 - DNA, 20 bp, Artificial Sequence
  NSE reverse outer primer
gaaggggatcacagcacact

SEQ ID NO: 26 - DNA, 20 bp, Artificial Sequence
  NSE reverse inner primer
ctgattgaccttgagcagca

SEQ ID NO: 27 - DNA, 20 bp, Artificial Sequence
  GFAP forward outer primer
gaggcagaagctccaagatg

SEQ ID NO: 28 - DNA, 20 bp, Artificial Sequence
  GFAP forward inner primer
agaacaacctggctgcgtat

SEQ ID NO: 29 - DNA, 20 bp, Artificial Sequence
  GFAP reverse common primer
cggcgatagtcgttagcttc

SEQ ID NO: 30 - DNA, 72 bp, Artificial Sequence
  synthetic double-stranded oligonucleotide for lentivirus-based expression of shRNA
cgctgcagttgccaacttgtcaatgaattcaagagattcattgacaagttggcaatttttgatatctagaca

SEQ ID NO: 31 - DNA, 20 bp, Artificial Sequence
  forward primer for cloning of mBest1 cDNA fragment
accttcaacatcagcctaaa

SEQ ID NO: 32 - DNA, 20 bp, Artificial Sequence
  reverse primer for cloning of mBest1 cDNA fragment
ctttctggtttttctggttg

(19) United States
(12) Patent Application Publication
(10) Pub. No.: US 2011/0201668 A1
(43) Pub. Date: Aug. 18, 2011

(54) REGULATION OF NEUROTRANSMITTER RELEASE THROUGH ANION CHANNELS

(75) Inventors: Justin Changjoon Lee, Seoul (KR); Dong-Ho Woo, Seoul (KR); Hyung-Ju Park, Seoul (KR); Hye-Kyung Park, Yangsan-si (KR)

(73) Assignee: KOREA INSTITUTE OF SCIENCE AND TECHNOLOGY, Seoul (KR)

(21) Appl. No.: 12/865,126

(22) PCT Filed: Jan. 30, 2008
(86) PCT No.: PCT/KR2008/000564

§ 371 Date: Dec. 15, 2010

(51) Int. Cl. A61K 31/7088; C07H 21/02; C12N 15/63; C12Q 1/02; C12Q 1/68

(52) U.S. Cl. 514/44 A; 536/24.5; 435/455; 435/29; 435/6.1

(57) ABSTRACT

A novel use of anion channels, preferably Ca2+-activated anion channels (CAACs), in regulating release of neurotransmitters from neurons and/or astrocytes is provided. More specifically, CAAC activity regulators, agents for regulating neurotransmitter release comprising such CAAC activity regulators, and methods of screening agents for regulating neurotransmitter release using CAAC as a target.

### CROSS REFERENCE TO RELATED APPLICATION

This application claims priority to and the benefit of Korean Patent Application No. 10-2008-0009377 filed on Jan. 30, 2008, which is hereby incorporated by reference for all purposes as if fully set forth herein.

### BACKGROUND OF THE INVENTION

(a) Field of the Invention

A novel use of anion channels, preferably Ca²⁺-activated anion channels (CAACs), in regulating release of neurotransmitters from neurons and/or astrocytes is provided. More specifically, CAAC activity regulators, agents for regulating neurotransmitter release comprising such CAAC activity regulators, and methods of screening agents for regulating neurotransmitter release using CAAC as a target.

(b) Description of the Related Art

Neurotransmitters, which transmit signals between a neuron and another neuron, are largely classified into four categories: amino acids (e.g., acetyl choline, glycine, aspartic acid, glutamate, and the like), amines (e.g., dopamine, adrenaline (epinephrine), noradrenalin, gamma-aminobutyric acid (GABA), and the like), peptides (e.g. vasopressin, and the like) and fatty acids (e.g. histamine, serotonin, and the like). Those chemicals are known to diffuse across the synapse to deliver information between the neurons. Since the neurotransmitters play a significant role in signal transmission between neurons, such transmissions can be effectively controlled by regulating neurotransmitter release.

Astrocytes provide structural scaffolding and nutrients to neurons as well as a mechanism for removing released neurotransmitters. Recently, several studies have shown that astrocytes can be activated by sensory stimulation or several pathological conditions including brain ischemia or inflammation. These stimuli evoke increases in intracellular Ca²⁺ in astrocytes, which in turn induce the release of active substances termed gliotransmitters. These released gliotransmitters are known to be involved in modulating neuronal synaptic plasticity and synaptic scaling, or even excitotoxicity.

Recent studies have suggested a novel role for astrocytes in the neuronal synaptic activation based on the finding that astrocytes can release gliotransmitters including excitatory amino acids (EAAs)—such as glutamate, which activates neuronal NMDA receptors. Although vesicular and non-vesicular mechanisms have been suggested as a system for controlling astrocytic glutamate release, exact molecular correlates in the activation mechanism remain unclear.

Similar to neurons, astrocytes have been suggested to release gliotransmitters through vesicle-dependent exocytosis. However, some cases of gliotransmitter release from astrocytes have recently been observed to occur which cannot be explained by vesicular exocytosis. This thus suggests a possibility that there is other channel for the release of gliotransmitters from astrocytes, than vesicular exocytosis.

As such, it is now required to clearly reveal the channel of neurotransmitters from neurons and/or astrocytes, in order to treat several pathological conditions modulated by the release of neurotransmitters including gliotransmitters—such conditions as associated with neuronal synaptic plasticity, synaptic scaling, excitotoxicity, and the like.

### SUMMARY OF THE INVENTION

In order to meet the needs stated above, the present invention is based on the present inventors' finding that Ca²⁺-activated anion channel (CAAC) plays a significant role in neurotransmitter release regulation occurring at neurons and/or astrocytes. In other words, the present invention aims to provide technology to prevent, treat, and reduce various pathological conditions resulting from over- or under-release of neurotransmitters, by controlling CAACs and thereby regulating neurotransmitter release therethrough.

In this regard, an embodiment of the present invention provides a novel use of CAAC in regulation of neurotransmitter release from neurons and/or astrocytes.

Another embodiment of the present invention provides an agent for regulating neurotransmitter release or neuroprotective agents, comprising a CAAC activity regulator.

Still another embodiment of the present invention provides a method of screening agents for regulating neurotransmitter release or neuroprotective agents using CAACs as a target.

### BRIEF DESCRIPTION OF THE DRAWINGS

FIGS. 1a-1k show that astrocytes express functional CAACs.

FIG. 1a shows a representative image for gramicidin-perforated patch clamp obtained from isolated cultured astrocytes (Scale bar=20 μm).

FIG. 1b shows representative simultaneous recordings for 30 μM TFLLR-induced Ca²⁺ transient and inward current, in the cultured astrocytes (Vₕ=−70 mV).

FIG. 1c shows TFLLR-induced Ca²⁺ and current responses in the Ca²⁺ free extracellular solution (n=5).

FIGS. 1d-1f show that TFLLR-induced Ca²⁺ and current responses were inhibited by a preincubation.

FIG. 1d shows inhibition results when the cells were preincubated with 100 nM Thapsigargin for 5 min (n=5).

FIG. 1e shows inhibition results when the cells were preincubated with 50 μM BAPTA-AM for 30 min (n=5).

FIG. 1f shows inhibition results when the cells were preincubated with 2 μM U73122 for 10 min (n=5).

TFLLR was applied at the time point denoted by ♦, with 10 s of application duration.

FIG. 1g shows that TFLLR-induced inward current responses was inhibited by 100 μM niflumic acid, where niflumic acid attenuated but did not completely block the TFLLR-induced increases in intracellular Ca²⁺ (n=5).

FIG. 1h shows that various anion channel blockers, such as 100 μM Niflumic acid, 100 μM flufenamic acid and 100 μM NPPB all blocked TFLLR-induced current. Each bar represents mean±s.e.m. (One way ANOVA with Tukey's post hoc test; *p<0.05 versus TFLLR-treated group).

FIG. 1i shows I-V curves for TFLLR-induced current responses with or without 100 μM niflumic acid treatment.

FIG. 1j shows that the I-V curves for current responses were altered by substituting chloride ion (150 mM NaCl) in the external bath with isethionate (150 mM Na-Isethionate).

FIG. 1k is a bar graph representing the mean±s.e.m. of the reversal potentials as observed for the TFLLR-induced current. Black and red bars represent the reversal potentials as measured for the NaCl (n=8) and Na-Isethionate (n=5)-containing bath solutions, respectively.

FIGS. 2a-2j show that permeability of astrocytic CAACs for glutamate increased with intracellular Ca²⁺ increasing.

FIGS. 2a-2c show I-V curves for different ions substituted in place for NaCl in the extracellular bath, the substituting ions being: I⁻ (a), F⁻ (b), and glutamate (Glu) (c).

FIG. 2d shows the shifts in the reversal potentials as obtained in the above experiments a-c, including the measurements obtained for isethionate and glutamate used in the extracellular baths.

FIGS. 2e-2f show I-V curves from whole-cell patch clamp measurements using pipette solution containing Cs-glutamate (e; CsGlu) or the bulky glutamate analogue Cs-PGCA (f; CsPGCA), wherein the left panel is for the I-V curves obtained before (black trace) and after (gray trace) niflumic acid treatment, and the right panel is for the I-V curve for the niflumic acid-sensitive component, which was obtained by subtracting the gray trace from the black trace (red trace).

FIG. 2g shows the averaged niflumic acid-sensitive current, in which the chemical structures of PGCA and glutamate are shown.

FIG. 2h shows the averaged evoked EPSP (eEPSP) before (control) and during the application of TFLLR (30 μM), wherein the right panel is for a superimposed trace of the two eEPSP.

FIG. 2i shows the averaged evoked EPSP (eEPSP) before (niflumic) and during the application of TFLLR (niflumic/TFLLR) in the presence of 30 μM of niflumic acid, wherein the right panel is for a superimposed trace of the two eEPSP.

FIG. 2j shows the area (%) of averaged eEPSPs as a time course with the application of TFLLR (blank circles) or with the treatment of niflumic/TFLLR (filled circles), at the left panel (mean±s.e.m). At the right panel of FIG. 2j, a bar graph appears for the area (%) of averaged eEPSPs (*p<0.05 versus control; unpaired t-test), where the decrease of eEPSP area as observed in the presence of niflumic acid and TFFLR is not statistically significant (p>0.05 versus control; unpaired t-test).

FIGS. 3a-3e show that mBest1 is an astrocytic Ca²⁺-activated anion channel.

FIG. 3a shows the results of RT-PCR analysis for the expressions of mouse bestrophin genes from the brain (whole brain) cDNA library and from cultured astrocytes (Astrocyte), where Beta actin gene was used as control.

FIG. 3b shows In situ hybridization (ISH) for an mBest1 specific probe, where the upper left and lower panels show coronal and sagittal section of ISH using antisense probe, respectively, and the upper right panel shows ISH using sense probe in coronal section,

FIG. 3c shows the result of a representative single cell RT-PCR analysis for an acutely dissociated neuron and astrocyte (the primers amplified were: neuron-specific enolase (NSE; N); glial fibrillary acidic protein (GFAP; G); and mBest1 (B)).

FIG. 3d shows the activation of CAACs in whole cell patch clamp configuration, in which the left panel shows a representative photomicrograph of HEK293T cells expressing GFP and mBest1 (Scale bar=20 μm). The middle upper panels show representative responses in the absence and presence of niflumic acid (100 μM) in the same cell where currents were elicited by voltage steps from −100 mV to +100 mV; the middle lower panels show representative currents elicited by voltage steps in the same HEK293T cells transfected with GFP alone. The right panel shows a bar graph representing the magnitude of the holding current recorded at −70 mV (mean±s.e.m; ***p<0.001, GFP versus mBest1, unpaired t-test.) In the figure, “−” and “+” indicate the absence or presence of Niflumic acid, respectively.

FIG. 3e shows representative photomicrographs of astrocytes expressing mBest1 shRNA and GFP (Scale bar=20 μm) at the left panel. At the middle panel, representative current recordings showing responses from astrocytes transfected with empty vectors or shRNA. At the right panel, a bar graph appears summarizing the averaged current amplitudes in each group as mean±s.e.m (One way ANOVA with Tukey's post hoc test; *p<0.001 versus shRNA group).

FIGS. 4a-4h show that astrocytes release glutamate through mBest1 channels.

At the upper panels of FIGS. 4a and 4b, schematics of the recording arrangement for the glutamate sniffer patch technique are shown. At the lower panel, representative images of HEK293T cells recorded under whole cell voltage clamp are shown (HEK293T cells expressing GluR1 (L497Y) and DsRED or mBest1 and EGFP. Scale bar=20 μm).

FIG. 4c shows representative recordings using the sniffer patch technique in mBest1 and GluR1 (L497Y) expressing HEK293T cell pairs (▴ indicates the time point of break-through during patch clamp experiments).

FIG. 4d shows representative recording traces of sniffer patch in naïve and GluR1 (L497Y) expressing HEK293T cell pairs, where the inserted panel shows a representative trace of full activation of GluR1 (L497Y) by bath treatment of 1 mM glutamate in the same cell.

FIG. 4e shows a bar graph summarizing the results of sniffer patch experiments by mean±s.e.m (*p<0.05, **p<0.01, One way ANOVA with Tukey's post test versus mBest1-expressing group).

FIGS. 4f-4h shows representative sniffer patch recordings for astrocytic intracellular Ca²⁺ and the currents of adjacent GluR1 (L497Y)-expressing HEK293T cells.

FIG. 4f shows the lentiviral expression of scrambled shRNA (scrambled).

FIG. 4g shows the lentiviral expression of mBest1 shRNA (sh-mBest1) (♦ shows the time point of TFLLR treatment), and the inserts at the bottom show the maximal response of GluR1 (L497Y) by treatment of 1 mM glutamate in the same cells.

FIG. 4h is a bar graph summarizing the results of sniffer patch experiments in FIGS. 4f and 4g by mean±s.e.m (*p<0.05 versus scrambled shRNA group; unpaired t-test).

### DETAILED DESCRIPTION OF THE PREFERRED EMBODIMENTS

A more complete appreciation of the invention, and many of the attendant advantages thereof, will be readily apparent as the same becomes better understood by reference to the following detailed description.

An embodiment of the present invention provides a novel use of anion channels, preferably, CAACs, in the regulation of neurotransmitter release from neurons and/or astrocytes. In concrete embodiments of the present invention, it is found that CAACs are functionally expressed in neurons and/or astrocytes, and function as a release channel for glutamate which is one of excitatory neurotransmitters, thereby confirming the role of CAACs as a channel for neurotransmitter release.

The neurotransmitters may refer to any chemicals involved in the transmission of neuro-electric signals, including any chemicals released from neurons and astrocytes. The neurotransmitters may be preferably excitatory neurotransmitters, for example, one or more selected from the group consisting of acetyl choline, aspartic acid, D-serine, glutamate, enkephalin, and histamine. Most of said materials are negatively charged small molecules (macroanions) with molecular weight of 1,000 Da or less. In one embodiment of the present invention, it is observed that glutamate, which is a representative of said small molecules, is released through anion channel. In light of the characteristics of channel-mediated release, the release of glutamate through anion channel is expected to be similarly applicable to other negatively charged molecules with similar size.

Said CAACs may include any anion channels existing on neuron and/or astrocytes whose activities are modulated by Ca²⁺. More specifically, said CAACs may be an anion channel that is permeable to various anions such as fluoride ion, bromide ion, chloride ion, iodine ion, and the like; and/or macro-anions such as negatively charged amino acids, isethionate, and the like. An embodiment of the present invention confirmed that glutamate, which is a representative excitatory neurotransmitter, is released through the CAACs encoded by Bestrophin 1 gene (Best1) that is expressed on astrocyte. Said Bestrophin 1 is a type of chloride ion channels, and used as a representative case for showing that CAACs is permeable to neurotransmitters. Said Bestrophin 1 gene may be mammal-, preferably rodent- or primate-originated one; for instance, it may be mouse Bestrophin 1 (mBest1) gene (NM_{—}011913, SEQ ID NO: 1) or human Bestrophin 1 (hBest1) gene (NM_{—}004183, SEQ ID NO: 2).

Based on the finding that CAACs permeable to neurotransmitters as described above, it may be suggested that release of excitatory neurotransmitters can be effectively controlled by regulation of CAACs existing on neurons and/or astrocytes. Therefore, an embodiment of the present invention provides methods of regulating release of excitatory neurotransmitter by regulating CAACs, and also provides agents for regulating release of excitatory neurotransmitter containing a regulator for controlling CAACs as an active ingredient.

For instance, over-release of excitatory neurotransmitters may be inhibited by inactivating CAACs, through which such excitatory neurotransmitters are released. In an embodiment of the present invention, CAACs can be inactivated by removing Ca²⁺ or lowering Ca²⁺ concentration by treating with any known Ca²⁺ removal agent, Ca²⁺ level lowering agents, and the like. In another embodiment of the present invention, CAACs can be inactivated by any known anion-channel blocking agents. In still another embodiment of the present invention, CAACs can be inactivated by treating with short hairpin RNA (shRNA) against CAAC-coding nucleotide sequences and thereby suppressing the expression of CAACs at neurons and/or astrocytes.

Therefore, an embodiment of the present invention provides a method of inhibiting excitatory neurotransmitter release by inactivating CAACs on neurons and/or astrocytes using any conventional method known to the relevant arts. Another embodiment of the present invention provides an agent for inhibiting release of excitatory neurotransmitters, containing one or more selected from the group consisting of known Ca²⁺ removal agents, Ca²⁺ level lowering agents, anion channel blocking agents, and antisense RNAs or shRNAs against CAAC-coding nucleotides, as an active ingredient. For more effective regulation of anion channel activity, said agent for inhibiting excitatory neurotransmitters may include one or more selected from the group consisting of anion channel blocking agents and antisense RNAs or shRNAs against CAAC-coding nucleotides, as an active ingredient, with or without one or more selected from the group consisting of known Ca²⁺ removal agents, and Ca²⁺ level lowering agents.

Said Ca²⁺ removing agents, Ca²⁺ level lowering agents, and anion channel blocking agents may be any one conventionally known to the relevant art. For instance, said Ca²⁺ removing agent and/or Ca²⁺ level lowering agents may be, but not be limited to, calcium ion chelators such as BAPTA-AM, thapsigargin, phospholipase C inhibitor, and the like. Anion channel blockers may be, but not be limited to, niflumic acid, flumenamic acid, 5-nitro-2(3-phenylpropylamino)-benzoic acid (NPPB), 4,4′-diisothiocyanatostilbene-2,2′-disulfonic acid (DIDS), and the like.

Said CAAC-coding nucleotide may be a Bestrophin 1 (Best1) coding gene. Said Bestrophin 1 coding gene may be one selected from the group consisting of mammal-originated genes, preferably rodent- and primate-originated genes; for instance, it may be mouse Bestrophin 1 (mBest1) gene (NM_{—}011913, SEQ ID NO: 1) or human Bestrophin 1 (hBest1) gene (NM_{—}004183, SEQ ID NO: 2). Therefore, said antisense RNA against CAAC-coding nucleotide may be one corresponding to the DNA sequences of SEQ ID NO: 1 or SEQ ID NO: 2. In addition, said shRNA against said CAAC-coding nucleotide may be one or more selected from the group consisting of SEQ ID NO: 3 and SEQ ID NO: 4, as shown below.

(SEQ ID NO: 3)
5′-GATCCCCTTGCCAACTTGTCAATGAATTCAAGAGATTCA
TTGACAAGTTGGCAATTTTTA-3′,
(SEQ ID NO: 4)
3′-GGGAACGGTTGAACAGTTACTTAAGTTCTCTAAGTAACT
GTTCAACCGTTAAAAATTCGA-5′,

It is expected that various pathological conditions resulting from over-release of neurotransmitters can be treated and/or prevented by inhibiting over-release of neurotransmitters through CAACs. Therefore, an embodiment of the present invention provides neuroprotective agents that protect nerves from over-release of neurotransmitters, or compositions for preventing and treating various pathological conditions resulting from over-release of neurotransmitters, where the agents and compositions contain one or more selected from the group consisting of Ca²⁺ removal agents, Ca²⁺ level lowering agents, anion channel blocking agents, and antisense RNAs or shRNAs against CAAC-coding nucleotides, as an active ingredient. Another embodiment of the present invention provides methods of protecting nerves from over-release of excitatory neurotransmitters or methods of preventing and/or treating pathological conditions resulting from over-release of excitatory neurotransmitters, by inactivating CAACs on neurons and/or astrocytes.

Said neuroprotective agents or compositions for preventing or treating various pathological conditions resulting from over-release of excitatory neurotransmitters may include, as an active ingredient, one or more selected from the group consisting of anion channel blocking agents and antisense RNAs or shRNAs against CAAC-coding nucleotides, for more effectively regulating anion channel activity and controlling over neurotransmitter release. In addition, said neuroprotective agents or compositions for preventing or treating various pathological conditions resulting from over-release of excitatory neurotransmitters may still further include one or more selected from the group consisting of known Ca²⁺ removal agents and Ca²⁺ level lowering agents. The kinds of chemicals are as stated above which can be used as Ca²⁺ removal agents, Ca²⁺ level lowering agents, anion channel blocking agents, and antisense RNAs or shRNAs against CAAC-coding nucleotides. Said pathological conditions resulting from over-release of excitatory neurotransmitters may be memory-associated diseases (e.g., Alzheimer's disease, age-associated memory impairment, and the like), epileptic seizures, neurotransmitter-induced excitotoxicity, ischemia, brain stroke, brain hemorrhage, epilepsy, traumatic brain injury, hypoxia, and the like.

In another aspect of the present invention, neurotransmitter release can be promoted by activating CAACs, thereby promoting neurotransmission. Therefore, an embodiment of the present invention provides methods of promoting neurotransmitter release by activating CAACs on neurons and/or astrocytes, as well as agents for promoting neurotransmitter release containing CAACs activating agent as an active ingredient. Said CAAC activating agent may be any substance that is capable of directly or indirectly activating CAACs. For instance, the CAAC activating agents may be an agonist for G-protein coupled receptor (GPCR), such as peptide TFLLR and Bradykinin. Such agent to promote neurotransmitter release may have an effect on synaptic plasticity and thereby improving recognition, cognition, movement, memory, and/or learning capabilities. Thus the present invention provide compositions for improving recognition, cognition, motivation, memory, and/or learning capabilities, which comprise a CAAC activating agent as an active ingredient.

In another aspect, the present invention provides a novel use of Bestrophin 1 gene as a gene encoding CAAC that is a channel for release of neurotransmitters. Therefore, an embodiment of the present invention provides a method of constructing a channel for excitatory neurotransmitters on neurons and/or astrocytes, by using an expression vectors including Bestrophin 1 gene to express CAACs, which function as a channel for excitatory neurotransmitters in mammals, on neurons and/or astrocytes.

Still another aspect of the present invention provides a method of screening a novel neuroregulatory agent using CAACs on neurons and/or astrocytes as a target. More specifically, the screening method may include the steps of:

preparing a sample of neurons and/or astrocytes,

contacting said sample with a candidate substance,

testing whether or not CAACs on the neurons and/or astrocytes are activated; and

determining said candidate substance as a neurotransmission promoting agent when CAACs are activated, or determining said candidate substance as a neuroprotective agent when CAACs are not activated.

The CAAC activation as stated above can be verified by measuring the change in inward current in neurons and/or astrocytes after inactivating all other receptors and channels on neurons and/or astrocytes than CAAC. For instance, an increased inward current value after the treatment with a candidate substance indicates that CAACs have become activated, while a decreased inward current value after the treatment with the candidate substance indicates that CAACs have become inactivated. The methods of the inactivation of other receptors and channels on neurons and/or astrocytes than CAAC, and the measurement of the inward current, as described above, are widely known in the field to which the current invention belongs to, and thus, those skilled in the art are expected to apply the above methods at ease. For instance, the measurement of the inward current values may be conducted via the sniffer patch technique (Lee, C. J. et al. Astrocytic control of synaptic NMDA receptors. J Physiol 581, 1057-81 (2007), this document is incorporated hereto as a reference).

In the screening methods according to the current invention, the CAAC may be one encoded by Bestrophin 1 gene, which may have the sequence of SEQ ID NO: 1 or SEQ ID NO: 2. Said neurons and/or astrocytes may be originated from mammals, or preferably, from rodents or primates.

The methods of regulation on neurotransmitter release according to the present invention may be beneficially applicable for the prevention or treatment of diseases associated with over-release of neurotransmitter, or for the improvement of recognition, cognition or learning capabilities related to synaptic plasticity.

The present invention is further explained in more detail with reference to the following examples. These examples, however, should not be interpreted as limiting the scope of the present invention in any manner.

### Example 1

### Example 1: Culture of HEK293T Cells and Astrocytes of Cortex of

### Mouse Brain

1.1. mBest1 Cloning

For the cloning of full-length mouse bestrophin 1 (mBest1) cDNA, total RNA was purified from cultured astrocytes or testis from adult male mice (C57BL/6), and cDNA was synthesized using Super Script III reverse transcriptase (Invitrogen) and amplified by PCR using 21 by primers starting and ending coincident with the open reading frame sequences based on NCBI database cDNA [GenBank accession numbers NM_{—}011913 XM_{—}129203, SEQ ID NO: 1]. Resulting PCR products were cloned into a pGEM-T easy vector (Promega) and sequenced.

The RT-PCR primers used to check expression of mBest1, 2, and 4 from cDNA were as followings:

mBest1-F:
(SEQ ID NO: 5)
5′-aggacgatgatgattttgag-3′,
mBest1-R:
(SEQ ID NO: 6)
5′-ctttctggtttttctggttg-3′;
mBest2-F:
(SEQ ID NO: 7)
5′-TCGTCTACACCCAGGTAGTC-3′,
mBest2-R:
(SEQ ID NO: 8)
5′-GAAAGTTGGTCTCAAAGTCG-3′;
mBest4-F:
(SEQ ID NO: 9)
5′-AAAGGCTACGTAGGACATGA-3′,
mBest4-R:
(SEQ ID NO: 10)
5′-GAAAGGACGGTATGCAGTAG-3′.

To test the presence of other CAAC candidate in mouse brain or astrocyte, following primer sets were used:

mCLCA1, 2, 4-F:
(SEQ ID NO: 11)
5′-TTCAAGATCCAAAAGGAAAA-3′,
mCLCA1, 2, 4-R:
(SEQ ID NO: 12)
5′-GCTCAGTCTGGTTTTGTTTC-3′,
mCLCA5-F:
(SEQ ID NO: 13)
5′-TAAGATTCCAGGGACAGCTA-3′,
mCLCA5-R:
(SEQ ID NO: 14)
5′-AAAGGAGGAAAAATACCTGG-3′,
mTtyh1-F:
(SEQ ID NO: 15)
5′-AGACACCTATGTGCTGAACC-3′,
mTtyh1-R:
(SEQ ID NO: 16)
5′-AGAAAAGAGCATCAGGAACA-3′,
mTtyh2-F:
(SEQ ID NO: 17)
5′-CCAGCTTCTGCTAAACAACT-3′,
mTtyh2-R:
(SEQ ID NO: 18)
5′-AATCTCTGTCCCTGTTGATG-3′,
mTtyh3-F:
(SEQ ID NO: 19)
5′-CAGTACTGAGTGGGGACATT-3′,
mTtyh3-R:
(SEQ ID NO: 20)
5′-CTGTGACAAAGGAGAAGAGG-3′.

For single cell PCR, a single astrocyte and neuron was acutely and mechanically dissociated from cortex of adult mouse brain, and cDNA of single, dissociated cell was amplified using Sensicript RT kit (Qiagen). Neuron-specific enolase (NSE, 300 bp)) and glial fibrillary acidie protein (GFAP, 360 bp) were used to identify the harvested cell type. In single cell PCR amplification was performed using the following primers:

mBest1 forward outer primer:
(SEQ ID NO: 21)
5′-aggacgatgatgattttgag,
mBest1 forward inner primer:
(SEQ ID NO: 22)
5′-accttcaacatcagcctaaa,
mBest1 reverse common primer:
(SEQ ID NO: 23)
5′-ctttctggtttttctggttg,
NSE forward common primer:
(SEQ ID NO: 24)
5′-gctgcctctgagttttaccg,
NSE reverse outer primer:
(SEQ ID NO: 25)
5′-gaaggggatcacagcacact,
NSE reverse inner primer:
(SEQ ID NO: 26)
5′-ctgattg accttgagcagca,
GFAP forward outer primer:
(SEQ ID NO: 27)
5′-gaggcagaagctccaagatg,
GFAP forward inner primer:
(SEQ ID NO: 28)
5′-agaacaacctggctgcgtat,
GFAP reverse common primer:
(SEQ ID NO: 29)
5′-cggcgatagtcgttagcttc.

The first PCR amplification was performed as described below. Samples were heated to 94° C. for 5 min. Each cycles consisted of denaturation at 94° C. for 30 sec, annealing at 50° C. for 30 sec, and elongation at 72° C. for 30 sec. Forty-two cycles were performed with a programmable thermocycler (Eppendorf). The second PCR condition consisted of denaturation at 94° C. for 30 sec, annealing at 55° C. for 30 sec, and elongation at 72° C. for 30 sec for forty-two cycles. After all PCR cycles were complete, the samples were heated to 72° C. for 10 min and subsequently cooled to 4° C. until analysis.

1.2. Plasmid Construction of mBest1 and Expression

In order to express mBest1 in mammalian cells, an mBest1 full-length fragment from pGEM-T easy plasmid (6.65 kb, Promega) was subcloned into pcDNA 3.1 (Invitrogen) by HindIII site and NotI site. The plasmid constructs were transfected into HEK293T cells (ATCC) using Effectene transfection reagent (Qiagen). To carry out whole cell patch clamp recordings, 1.5˜2 μg of plasmid, which was obtained by cloning mBest1 in cDNA extracted from mouse brain or cultured astrocytes, and then, subcloning into pcDNA3.1 plasmid (Invitrogen), plus pEGFP-N1 (Clontech) were used to transfect one 35 mm culture dish. One day after transfection, cells were replated onto glass coverslips for electrophysiological recording. Transfected cells were identified by EGFP fluorescence and used for patch clamp experiments within 24-36 hrs.

1.3. mBest1 shRNA and Lentivirus Production

For plasmid-based shRNA expression, the following complementary oligonucleotides were annealed and inserted into the HindIII/BglII sites of pSUPER-GFP vector (Oligo Engine):

(SEQ ID NO: 3)
5′-GATCCCCTTG CCAACTTGTC AATGAATTCA AGAGATTCAT
TGACAAGTTG GCAATTTTTA-3′
(SEQ ID NO: 4)
3′-GGGAACGGTTGAACAGTTACTTAAGTTCTCTAAGTAACTGTTC
AACCGTTAAAAATTCGA-5′,

(corresponding to nucleotide sequence of mBest1 (563-582)).

The efficacy of the construct to interfere with mBest1 expression was tested against heterologously expressed mBest1 in HEK293T cells (ATCC) by measuring specific CAAC currents. For lentivirus-based shRNA expression, lentiviral vector containing mBest1 gene was constructed by inserting synthetic double-strand oligonucleotides 5′-CGCTGCAGTTGCCAACTTGTCAATGAATTCAAGAGATTCATTGACAAGTT GGCAATTTTTGATATCTAGACA-3′ (SEQ ID NO: 30) into pstI-XbaI restriction enzyme sites of shLenti2.4 CMV lentiviral vector (Macrogen) and verified by sequencing. Scrambled oligonucleotides inserted shLenti construct (Macrogen) was used as control. The production of lentivirus was performed by Macrogen Inc. as described earlier (Dull, T. et al., A third-generation lentivirus vector with a conditional packaging system. J Virol 72, 8463-71 (1998), which is hereby incorporated by reference for all purposes as if fully set forth herein).

1.4. In Situ Hybridization

To make specific riboprobe for mRNA of mBest1, the present inventors cloned partial cDNA fragments of mBest1 using RT-PCR with mouse cultured cortical astrocytes. Primers used were as follows:

forward:
(SEQ ID NO: 31)
5′-ACCTTCAACATCAGCCTAAA-3;
reverse:
(SEQ ID NO: 32)
5′-CTTTCTGGTTTTTCTGGTTG-3′.

The plasmid was linearized and used for in vitro transcription (Roche Dignostics) to label RNA probes with digoxigenin-UTP. In situ hybridization was performed as previously described with some modifications. Frozen brains of adult mouse brains were sectioned at 20 m thicknesses on a cryostat. The sections were then fixed in 4% paraformaldehyde, washed with PBS, and acetylated for 10 min. The sections were incubated with the hybridization buffer (50% formamide, 4×SSC, 0.1% CHAPS, 5 mM EDTA, 0.1% Tween-20, 1.25×Denhartdt's, 125 ug/ml yeast tRNA, 50 ug/ml Heparin) and digoxigenin-labeled probes (200 ng) for 18 h at 60° C. Non-specific hybridization was removed by washing in 2×SSC for 10 min and in 0.1×SSC at 50° C. for 15 min. For immunological detection of digoxigenin-labeled hybrids, the sections were incubated with anti-digoxigenin antibody conjugated with alkaline phosphatase (Roche Diagnostics) for 1 h, and the color reaction was carried out with 4-nitroblue tetrazolium chloride/bromo-4-chloro-3-indolyl phosphate (NBT/BCIP; Sigma). Sections were dehydrated and mounted with Vectamount (Vector Laboratory).

### Example 2

### Measurement of Ca²⁺ and Glutamate

2.1. Recording Solutions for Simultaneous Ca²⁺ Imaging and Perforated Patch Clamp Recording

The External solution was comprised of (in mM) 150 NaCl, 10 HEPES, 3 KCl, 2 CaCl₂, 2 MgCl₂, 5.5 glucose, at pH 7.3 (˜320 mOsm). For voltage clamp recordings, the internal solution contained 25 μg/ml gramicidin D and (in mM) 75 Cs₂SO₄, 10 NaCl, 0.1 CaCl₂, and 10 HEPES, at pH 7.1 (˜310 mOsm). For current clamp recordings, the internal solution contained 25 μg/ml gramicidin D and (in mM) 75 K₂SO₄, 10 KCl, 0.1 CaCl₂, and 10 HEPES, at pH 7.1 (˜310 mOsm). Pipette resistances ranged from 5 to 8 M. For perforated patch clamp, it took 20 to 30 min to achieve acceptable perforation, with final series resistances ranging from 15 to 40 MΩ.

2.2. Whole-Cell Patch Clamp

Patch pipettes which have 3-6MΩ of resistance are filled with the standard intracellular solution. Current voltage curves were established by applying 100- or 200-ms-duration voltage ramps from −100 to +100 mV. The ramp duration was 10 s. Data were acquired by an Axopatch 200A amplifier controlled by Clampex 9.0 via a Digidata 1322A data acquisition system (Molecular Devices). Experiments were conducted at room temperature (20˜24° C.). The standard pipette solution was comprised of (in mM) 146 CsCl, 2 MgCl₂, 5 (Ca²⁺)-EGTA, 8 HEPES, and 10 sucrose, at pH 7.3, adjusted with CsOH. The concentration of free [Ca²⁺]i in the solution was determined (Kuruma, A. & Hartzell, H. C. Bimodal control of a Ca(²⁺)-activated Cl(−) channel by difference Ca(²⁺) signals. J Gen Physiol 115, 59-80 (2000), which is hereby incorporated by reference for all purposes as if fully set forth herein). The standard extracellular solution was comprised of (in mM) 140 NaCl, 5 KCl, 2 CaCl₂, 1 MgCl₂, 15 glucose, and 10 HEPES, with pH 7.3 as adjusted using NaOH.

2.3. Measurement of Glutamate Permeability by Sniffer Patch

The sniffer patch technique, which is used for determining whether or not one is permeable to glutamate, was performed as described in Lee, C. J. et al. Astrocytic control of synaptic NMDA receptors. J Physiol 581, 1057-81 (2007), which is hereby incorporated by reference for all purposes as if fully set forth herein.

To test whether mBest1 channel was permeable to glutamate, the present inventors tested two kinds of experimental pairs.

1. In experiments using HEK293T-HEK293T cell pairs of mBest1 (with GFP), the sniffer patch technique used as a glutamate source the mBest1 or GluR1 (L497Y) (with DsRED)-expressing cell; and as a detector the GluR1 (L497Y) (with DsRED)-expressing cell. After obtaining gigaohm seal of both pipettes onto the two adjacent cells, the GluR1 (L497Y)-expressing detector cell was firstly ruptured, and then counterpart glutamate source HEK293T cell was ruptured using pipette containing 4.5 μM of Ca²⁺ and 145 mM glutamate (in mM: 145 CsGlutamate, 5 Ca-EGTA-NMDG, 2 MgCl₂, 10 HEPES, 10 Sucrose, pH 7.3).

2. In experiments using astrocyte-HEK293T cell pairs, the sniffer patch techniques used naïve, scrambled- or mBest1-specific shRNA expressing (with GFP) astrocytes as a glutamate source; and GluR1 (L497Y) expressing HEK293T cells (with DsRED) as a detector. After obtaining gigaohm sealing, GluR1 (L497Y)-expressing cell was firstly ruptured, and then counterpart astrocytes were pressure-applied with 500 uM of TFLLR to evoke an increase in astrocytic intracellular Ca²⁺ and resulting glutamate release onto the adjacent HEK293T cells.

GluR1LY-expressing detector cells were patched with the pipette solution pH 7.3 containing 110 mM CsGluconate, 30 mM CsCl, 5 mM HEPES, 4 mM NaCl, 2 mM MgCl₂, 5 mM EGTA, and 1 mM CaCl₂. The percentage of GluR1 (L497Y)-mediated current to the full activation level was analyzed by dividing the current amplitude of GluR1 (L497Y) current obtained through sniffer patch measurement by that of fully activated GluR1 (L497Y) current in the same cells.

### Example 3

### Verification of Functional Expression of CAACs in Astrocytes

Astrocytic Gq-coupled receptors such as P2Y receptor, bradykinin receptor, and protease activated receptor-1 (PAR-1) are known to induce a transient increase in the intracellular Ca²⁺ concentration ([Ca²⁺]i), which in turn leads to glutamate release from astrocytes by a Ca²⁺ dependent mechanism. The present inventors have previously shown that glutamate release in this fashion from astrocytes strengthens the synaptic NMDA receptor function by relieving Mg²⁺-dependent pore block of NMDA receptors (Lee, C. J. et al. Astrocytic control of synaptic NMDA receptors. J Physiol 581, 1057-81 (2007). However, the mechanism by which PAR1 activation facilitates glutamate release following an increase of astrocytic [Ca²⁺]i has not been known. Therefore, using PAR1 activation as a tool for selective induction of astrocytic [Ca²⁺]i increase, the inventors investigated the Ca²⁺-dependent downstream processes leading to glutamate release, in order to identify any molecular correlates in the release mechanism.

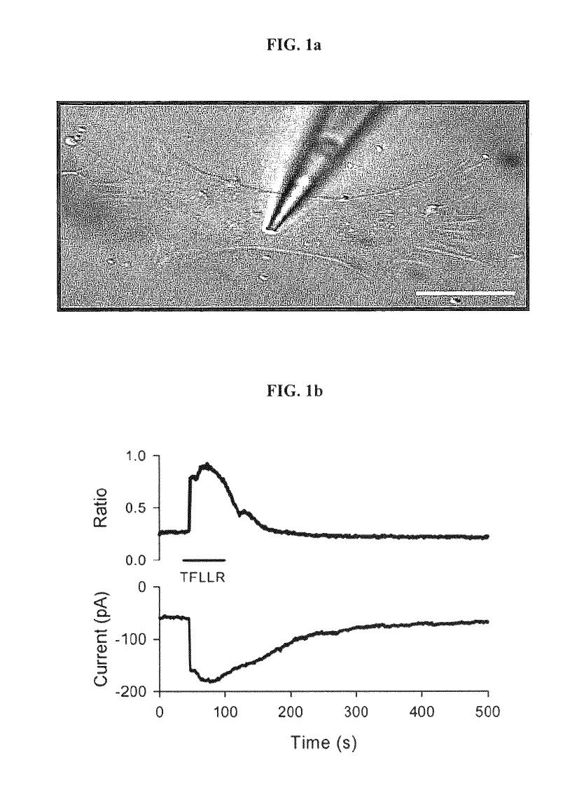

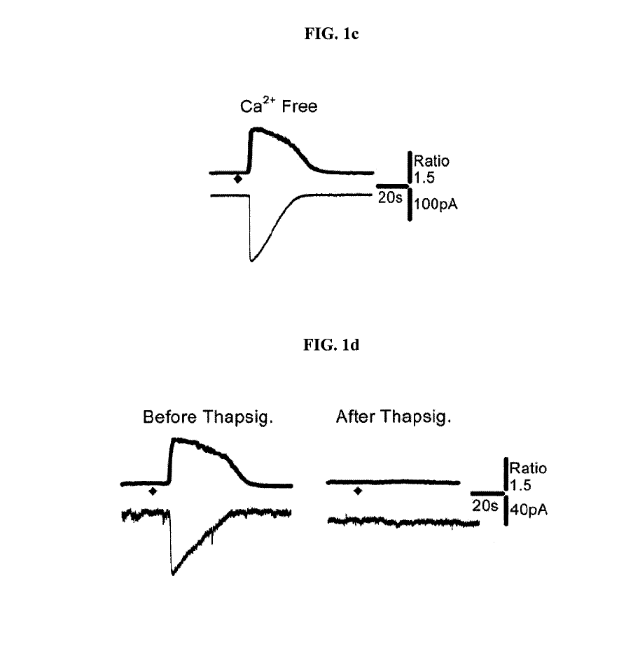

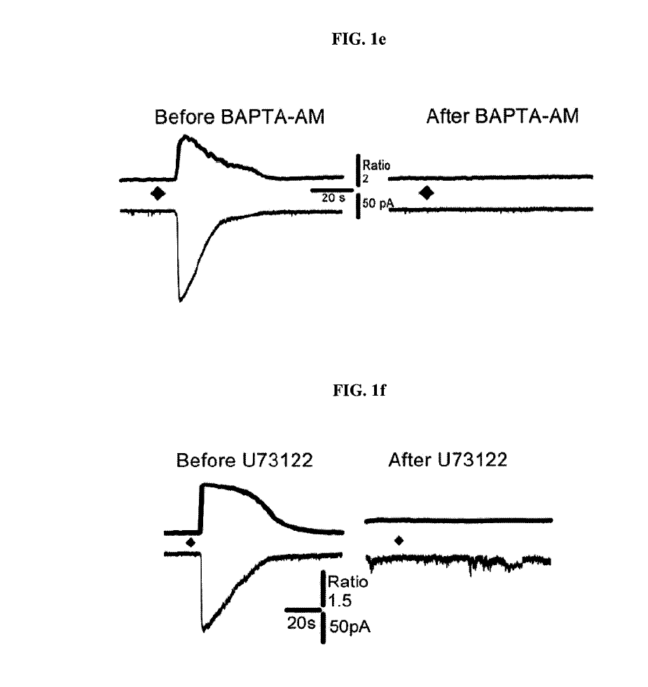

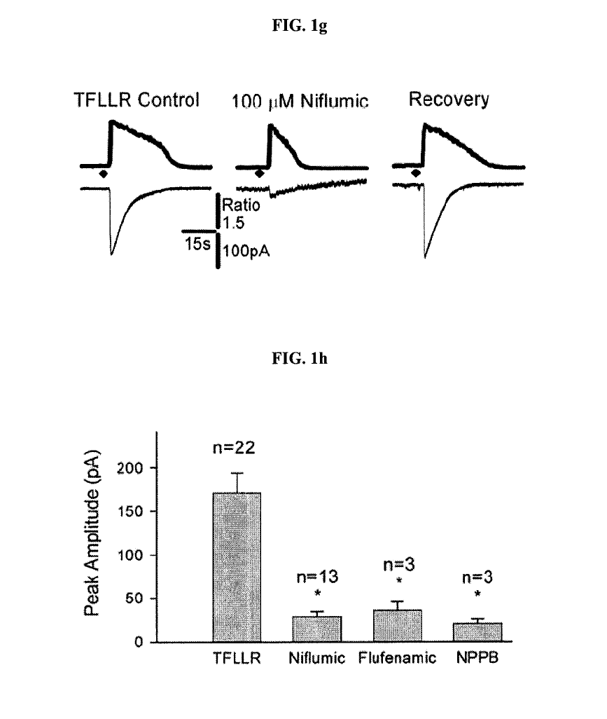

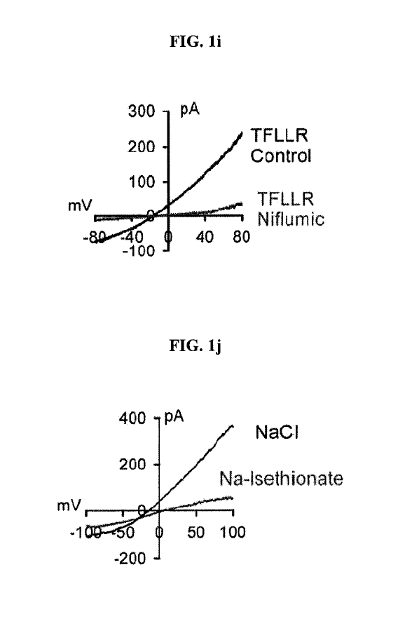

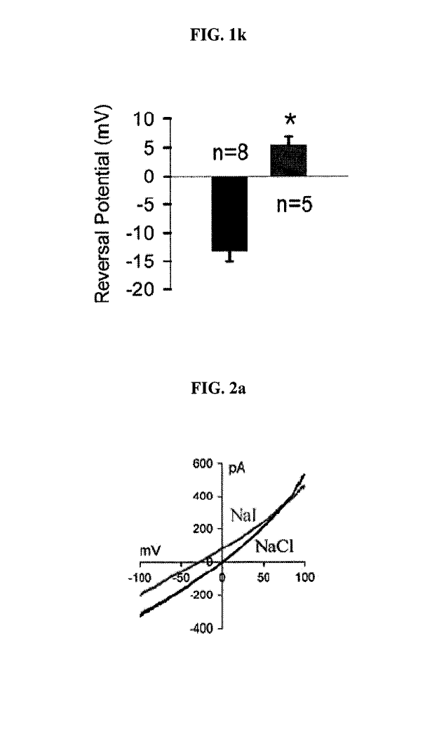

A recent report demonstrated that glutamate release from cultured astrocytes following PAR activation was inhibited by anion channel blockers, suggesting an involvement of anion channels. To test if activation of PAR1 by its specific agonist (e.g., TFLLR) causes any change in membrane conductance that might contribute to glutamate release from astrocytes, the whole cell currents and intracellular Ca²⁺ responses in cultured astrocytes under gramicidin-D perforated patch configuration were simultaneously recorded (FIG. 1a). This technique minimized dialysis of intracellular ions. Following the application of 30 μM TFLLR (˜3-fold of EC₅₀), the present inventors observed the current inactivated with lapse of time allowed for the Ca²⁺ responses (154±16 pA, n=26; FIG. 1b).

When other types of Gq-coupled receptors such as P2Y receptor, bradykinin receptor, lysophosphatidic acid (LPA) receptor, and prostaglandin E2 (PGE2) receptor were activated by corresponding selective agonists, concomitant increases of [Ca²⁺]i and inward current were similarly observed, indicating that this current induction is a general mechanism shared by a host of astrocytic Gq-coupled receptors.

Such TFLLR-induced current was intact in the Ca²⁺ free bath (FIG. 1c). However, BAPTA-AM treatment (chelation) eliminated both the TFLLR-induced [Ca²⁺]i transient and current (FIG. 1e), indicating that the TFLLR-induced current is dependent on intracellular Ca²⁺. Impairment of the Ca²⁺ release from internal stores by application of either thapsigargin (Tocris, 100 nM, FIG. 1d) or the phospholipase C inhibitor, U73122 (Tocris, 2 μM, FIG. 1f), reduced both the TFLLR-induced [Ca²⁺]i increase and the inward current. In addition, this [Ca²⁺]i-activated current was also blocked by niflumic acid (100 μM), flufenamic acid (100 μM), and NPPB (100 μM) (FIGS. 1g and 1h), all well-known inhibitors of Ca²⁺-activated anion channels (CAACs). Niflumic acid-mediated block of the TFLLR-induced current was voltage-independent (FIG. 1i), with an IC₅₀ value of 9.8 μM, which is virtually identical to the reported IC₅₀ value for CAACs expressed in Xenopus laevis oocytes (IC₅₀=10.1 μM).

Subsequently, it was tested whether the astrocytic PAR1-activated inward current was carried in part by Cl⁻. The inventors determined the current-voltatge (I-V) relationship for the TFLLR-induced current in the presence of 150 mM NaCl in external solution and compared it to the I-V curve obtained in the presence of 150 mM Na+-isethionate (FIG. 1j). The reversal potential was significantly shifted to the right (from −13.2±1.9 to +5.4±1.5 mV, n=8, and 5, respectively; p<0.05) by substitution of Cl— with isethionate (FIG. 1k), suggesting that Cl— carried a portion of the current and that isethionate was less permeable than Cl—. In a separate experiment, the reversal potential of the TFLLR-induced current under whole-cell configuration was about −71±1.5 mV (n=4), which is consistent well with the calculated reversal of −75 mV, according to the Nernst equation for 7 mM internal Cl— (CsGluconate internal solution). Together, these data suggest that astrocytic CAACs are activated by an increase in cytosolic Ca²⁺ upon PAR-1 activation. In contrast, treatment of astrocytes with carbenoxolone (100 μM, Sigma) or chlorotoxin (1 μM, Sigma) did not block the current by CAACs, suggesting that hemi-channels or chlorotoxin-sensitive chloride channels are not involved in this Ca²⁺-activated Cl— flux. These results demonstrate for the first time the functional expression of CAACs in astrocytes.

### Example 4

### Test of Glutamate Permeability of CAAC

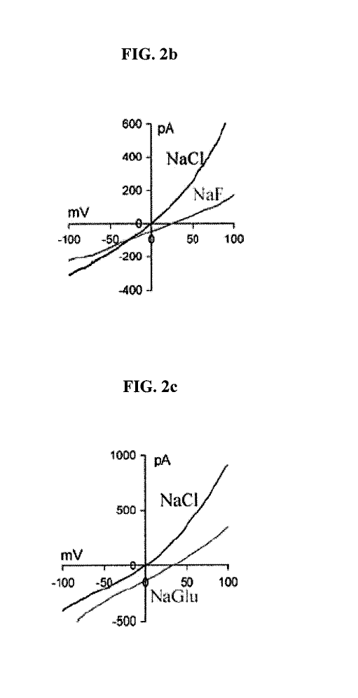

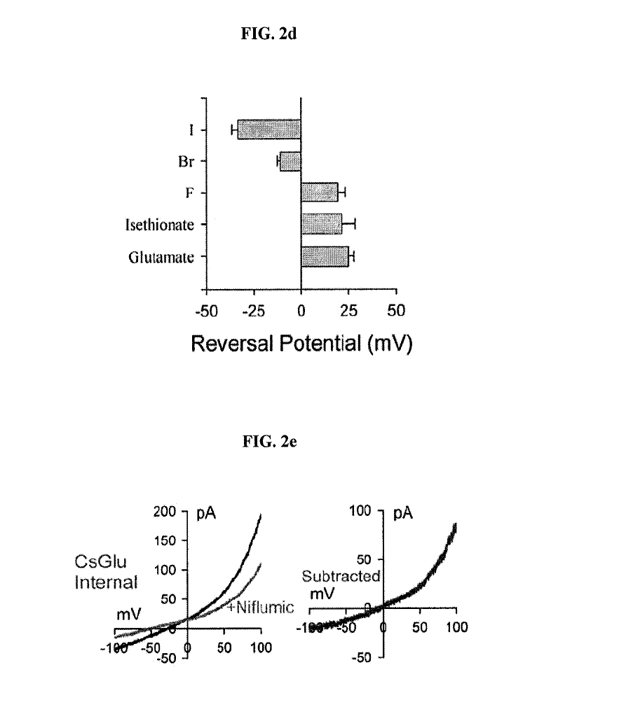

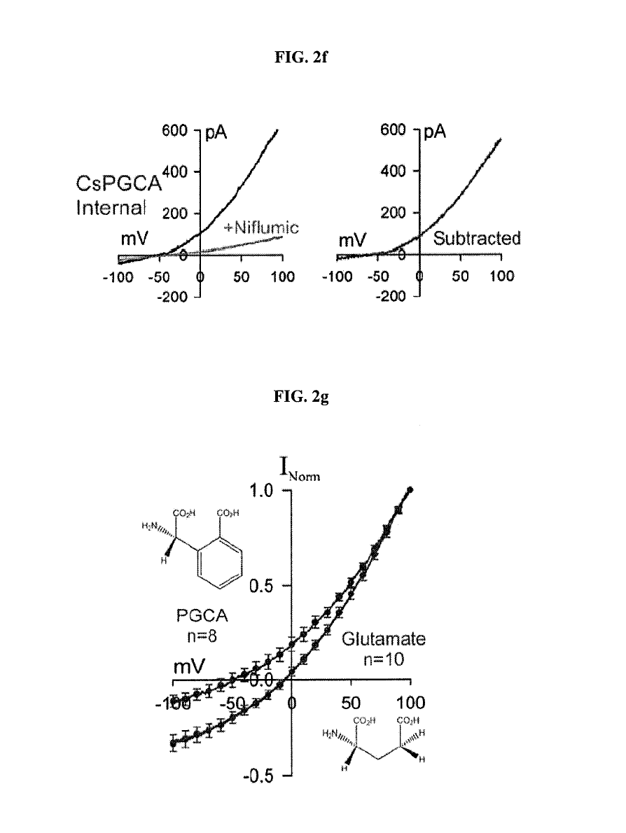

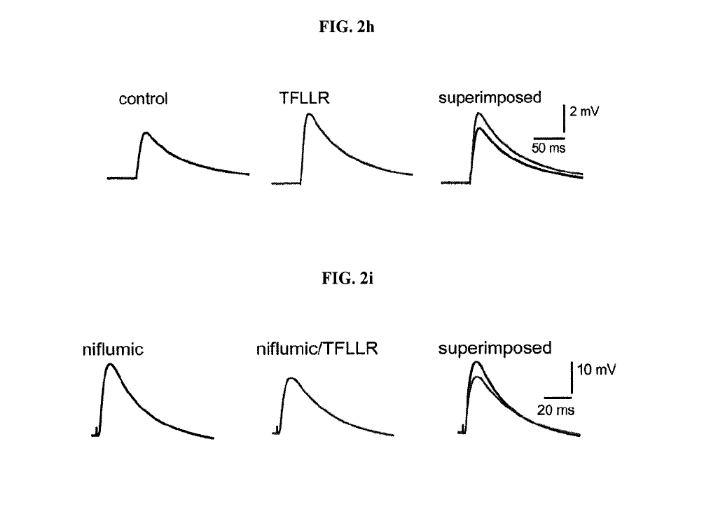

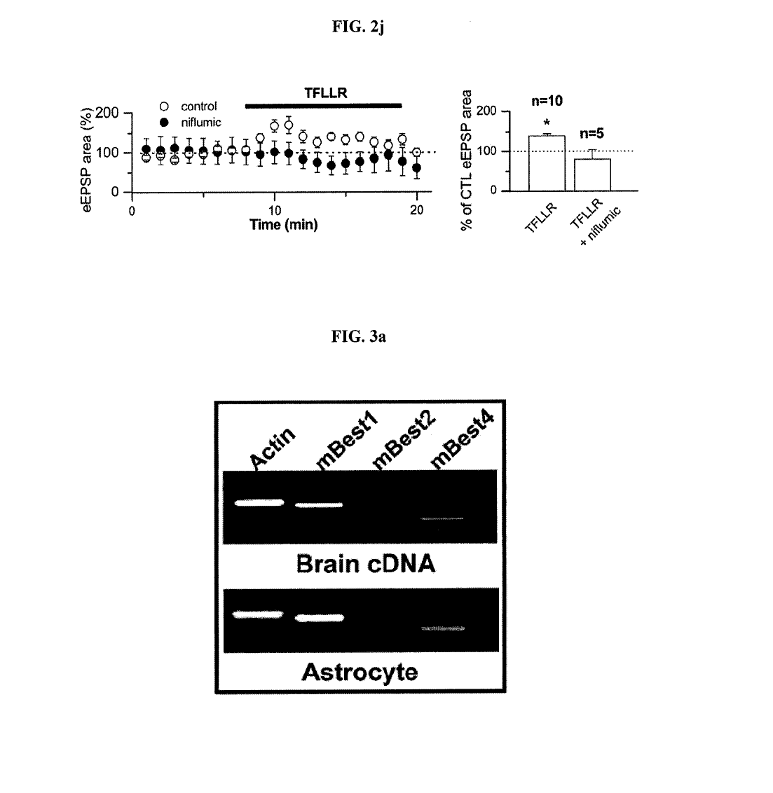

Because CAACs can permeate large anions and could be directly activated by applying internal solutions with known Ca²⁺ concentrations, it was tested whether astrocytic CAACs can permeate glutamate by directly applying internal solutions containing 4.5 μM of Ca²⁺ at which level CAACs are maximally activated (FIG. 10). It was found that direct activation of astrocytic CAACs displayed a non-desensitizing CAAC current, which was readily blocked by treatment of niflumic acid (FIG. 6c). In a series of ion substitution experiments in which different anions of external bath solution were used, the present inventors found that the I-V relationship of astrocytic CAACs was outwardly rectifying and displayed the permeability order of I>Br⁻>Cl⁻>F⁻ (FIG. 2a-2c), which was identical to the previously known properties of other CAACs reported in Xenopus oocytes and mammalian cells.

Surprisingly, substitution of Cl— ions with larger anions such as glutamate or isethionate also induced a significant outward current (anion influx) at very positive potentials (FIG. 2c and d), indicating that glutamate is permeable from outside to inside of the cells through CAACs. To examine the possibility of glutamate efflux from inside to outside of cells, the I-V relationship of the niflumic acid-sensitive component (FIG. 2e, right panel) of currents directly activated by 4.5 μM intracellular Ca²⁺ was measured. The inventors subtracted the I-V relationship obtained before (black trace) and after (gray trace) niflumic acid treatment (FIG. 2e, left panel). The measurement was conducted using an internal solution containing 4.5 μM of Ca²⁺ and glutamate (145 mM) as a sole anion. The inventors found a significant inward current at negative potentials, indicating an efflux of glutamate through CAACs (FIG. 2e and g; red trace). Replacing glutamate with phenylglycine-o-carboxylate, a conformationally restricted glutamate analogue with which a bulky aromatic ring fused into the glutamate backbone, showed a significantly reduced inward current (anion efflux) compared to glutamate (FIG. 2f, g). These data further support the possibility of glutamate conductance through CAACs. Moreover, TFLLR-evoked efflux of radiotracer from [³H]-glutamate-loaded cultures was significantly inhibited by 69% or 57% with the treatment of niflumic acid or flufenamic acid (n=11 and 4, respectively), which is consistent with the previous report.

The glutamate release through astrocytic CAACs was examined by using “sniffer-patch” technique and recording real-time glutamate release from cultured astrocytes (FIG. 10a). Using this technique, the present inventors observed that TFLLR-induced astrocytic glutamate release into an adjacent HEK293T cells expressing the non-desensitizing AMPA receptor subunit GluR1 (L497Y) mutant evoked an inward current sensitive to AMPA receptor antagonists, which is interpreted to reflect release of glutamate. The effects of TFLLR on current responses in transfected HEK293T cells was blocked by the treatment of niflumic acid (% block by niflumic acid=66.6±5.7%, FIGS. 10d to 100, which is consistent with the idea that astrocytic CAAC is permeable to glutamate. Taken together, these results indicate that the pores of astrocytic CAACs are large enough to allow glutamate permeation.

To assess whether CAAC-dependent glutamate release in astrocytes occurs and enhances synaptic potentials in vivo, a series of current clamp experiments were carried out to directly measure the effects of CAAC-dependent glutamate release on the evoked EPSPs (eEPSPs) of the Schaffer collateral to CA1 pyramidal neuron synapse in hippocampal slices. Under the similar recording conditions as previously described in ‘Lee, C. J. et al. Astrocytic control of synaptic NMDA receptors. J Physiol 581, 1057-81 (2007)’, it was found that TFLLR enhanced amplitudes and areas of evoked EPSPs (eEPSPs), which include a slow decay reflecting contribution of NMDA receptors. This enhancement of eEPSP is blocked by treatment with niflumic acid, suggesting that glutamate is released by permeation through astrocytic CAACs in vivo and modulates neuronal synaptic activities (FIGS. 2i-2k). Consistent with this, the present inventors also found that TFLLR-induced prolongation of slow, NMDA receptor (NMDAR)-mediated component of mEPSC decay as recorded under voltage clamp was also sensitive to the treatment with niflumic acid. Since niflumic acid minimally affected NMDAR-mediated current amplitude (FIG. 10), the blocking effect of niflumic acid on synaptic potentials or currents was unlikely due to a nonspecific effect on neuronal NMDARs.

### Example 5

### Test to Verify Whether mBest1 is an Anion Cannel Activated by Astrocytic Ca²⁺

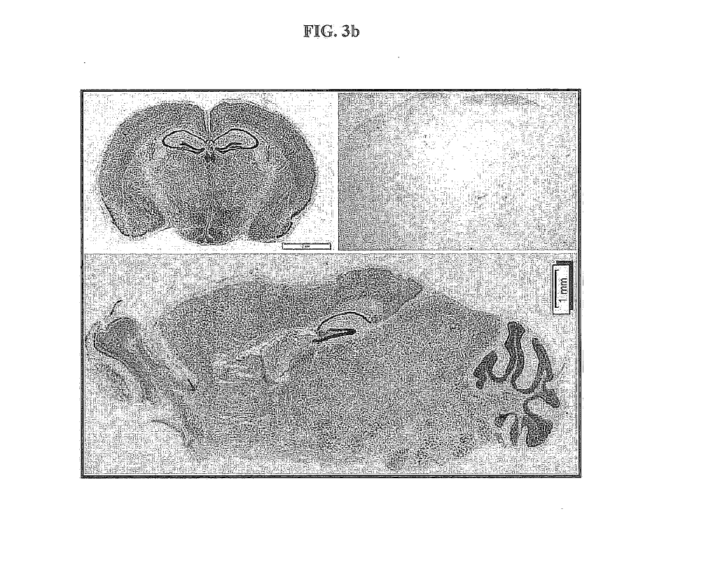

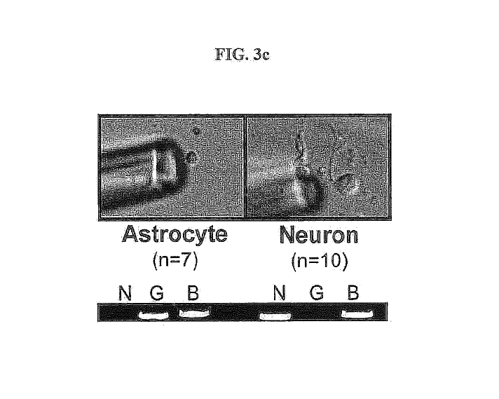

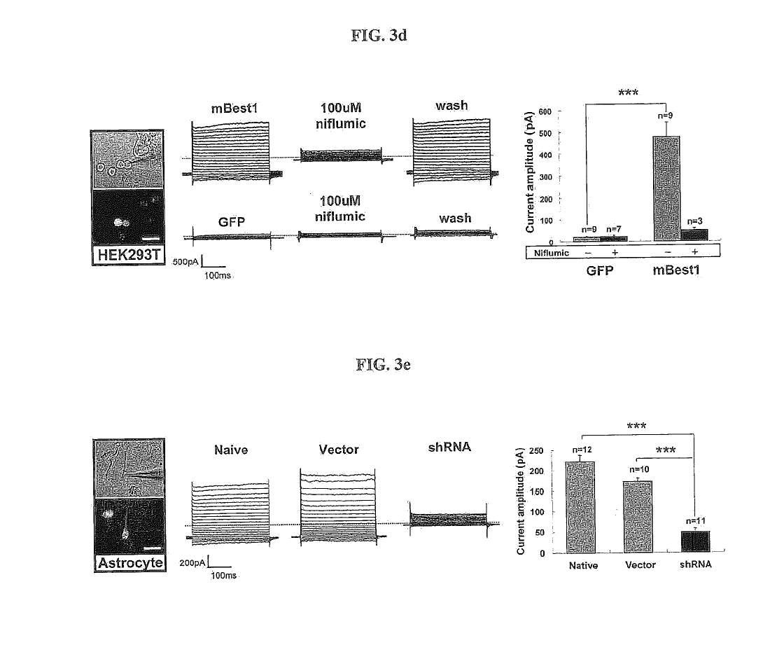

Molecular identification of CAACs has long remained unresolved and been hampered by the lack of specific blockers and an unambiguous assay system. In fact, CAACs are one of very few channels that have not yet been cloned. To identify the gene encoding CAAC in astrocytes, the present inventors performed reverse transcriptase polymerase chain reaction (RT-PCR) with primer sets for various candidate genes such as Cl— channel-Calcium Activated (CLCA), Drosophila tweety homolog (Ttyh), and bestrophin (Best) family genes, all of which have been suggested by others as CAACs. The above RT-PCR analysis demonstrated that mouse bestrophin 1 and 4 (mBest1 and 4) were expressed in brain and cultured astrocytes with much higher expression of mBest1 than mBest4, suggesting that mBest1 channel might account for the glutamate-permeable CAAC properties in astrocytes (FIG. 3a). In spite of significant expression of mouse Ttyh family genes in astrocytes (FIG. 11), these genes were not considered an astrocytic CAAC candidate in light of their recently reported properties of tweety channels—such as slow channel opening by cytosolic Ca²⁺, insensitivity to niflumic acid, and lack of outward rectification. Bestrophin channels are known to display similar properties of CAACs and are found to be expressed in peripheral tissues such as cilia of olfactory sensory neurons and retinal epithelial cells in which they are involved in olfactory transduction and retinal degeneration, respectively. However, until now both direct evidence of their expressions in the central nervous system and the role of astrocytic bestrophin channel have not been investigated yet. The present inventors firstly analyzed the expression pattern of mBest1 within brain regions and by cell types. In situ hybridization analysis showed a wide distribution pattern of mBest1 mRNA expression (FIG. 3b), suggesting that mBest1 serves a major role in the brain. Next, through the single-cell RT-PCR using mBest1-specific primer set and the cDNA of individual acutely-dissociated astrocyte or neuron from the adult mouse cortex, the present inventors has identified the expression of mBest1 in both GFAP (glial fibrillary acidic protein) and NSE (neuron specific enolase) expressing cell types (FIG. 3c), indicating that mBest1 is expressed not only in astrocytes but also in neurons.

To analyze whether mBest1 channels have similar properties to those of CAACs, the full-length mBest1 was cloned from both astrocyte and testis cDNAs and transiently expressed in HEK293T cells. It was found that mBest1-expressing HEK293T cells showed similar CAAC properties with those of astrocytes such as outward rectification, Ca²⁺-dependent channel activation, and sensitivity to niflumic acid (FIG. 3d and FIG. 11). By contrast, HEK293T cells transfected with GFP alone did not show any Ca²⁺ activated current. These data suggest that mBest1 is a possible molecular candidate for glutamate-permeable astrocytic CAACs.

Next, in order to determine the molecular identity of astrocytic CAACs as mBest1, the present inventors designed a mBest1-specific short hairpin RNA (shRNA) to selectively knock-down the expression of mBest1 and measure the effect of it on CAACs and ultimately on glutamate release from astrocytes. The specific and efficient knock-down of mBest1 channel by the shRNA was confirmed in HEK293T cells transfected with mBest1 cDNA (FIG. 12). Using this shRNA the present inventors found that CAAC current in astrocyte was significantly suppressed by mBest1-specific shRNA expression in astrocytes (FIG. 3e; naïve astrocytes: 221.3±16.4 pA, n=12; scrambled shRNA expressing astrocytes: 173.5±7.2 pA, n=10; mBest1 shRNA expressing astrocytes: 49.7±8.3 pA, n=11; One way ANOVA with Tukey's post hoc test; ***p<0.001 versus shRNA group). These results indicate that mBest1 encodes the majority of CAACs in astrocytes.

### Example 6

### Test of Glutamate Release Through mBest1 Channel

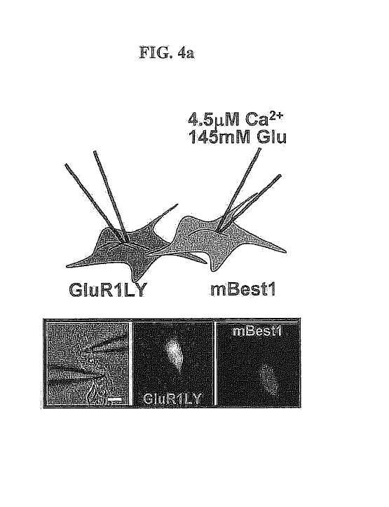

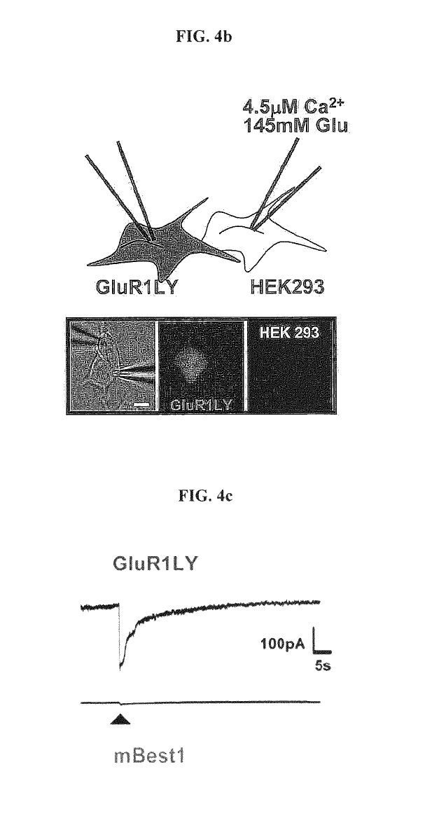

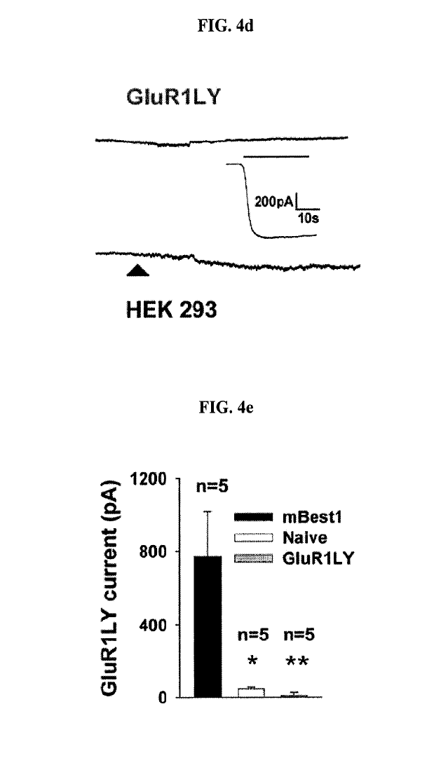

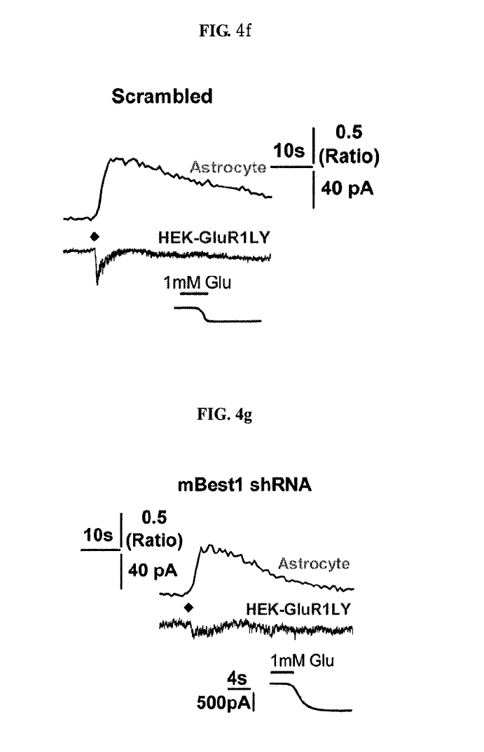

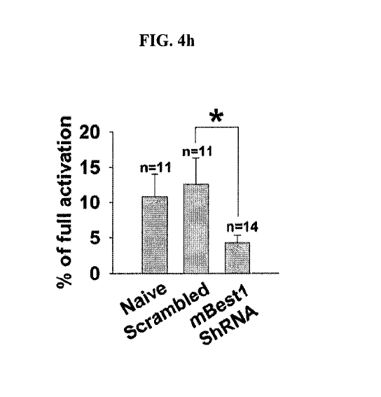

The release of glutamate through mBest1 channels was examined by using the sniffer-patch technique with patch pipette containing Ca²⁺ and glutamate to directly activate mBest1 channels upon membrane break-through (FIG. 4a, b). In the control experiment using HEK293T cells that are not expressing mBest1 or expressing GluR1 (L497Y), no detectable current was observed in the neighboring GluR1 (L497Y)-expressing HEK293T cells upon a break-through (naïve cells: 46.0±20.6 pA, n=5; GluR1 (L497Y) expressing cells: 8.0±3.6 pA, n=5). On the contrary, direct activation of the mBest1 channel was observed to induce significantly large amount of glutamate release from the mBest1-expressing HEK293T cells, indicating that glutamate permeates through mBest1 channels (FIGS. 4c and 4e; 770.0±344 pA, n=5, *p<0.05, **p<0.01, one way ANOVA with Tukey's post hoc test). These results directly established that mBest1 channels are required and working selectively for glutamate permeation.

Finally, in order to determine whether astrocytic mBest1 is responsible for Ca²⁺ dependent glutamate release, the present inventors performed sniffer-patch experiments between cultured astrocytes expressing scrambled shRNA or mBest1 shRNA, and GluR1 (L497Y)-expressing HEK293T cells. Glutamate release was significantly reduced at astrocytes by mBest1 shRNA but not by scrambled shRNA. As shown in FIGS. 4f-4i, the percentage of GluR1 (L497Y)-mediated current to the fully activated GluR1 (L497Y) current by 1 mM glutamate in naïve astrocytes was 10.8±3.2% (n=11); 12.7±3.6% (n=11) in the cells expressing scrambled shRNA; and 4.2±1.2%, n=14 in the mBest1 shRNA expressing cells (*p<0.05, scrambled vs. mBest1 shRNA, unpaired t-test. The increase in astrocytic intracellular Ca²⁺ was unaffected. These data strongly suggest that mBest1 channels contribute to Ca²⁺-dependent release of glutamate by direct permeation. The remaining component of glutamate release from astrocytes by mBest1 knock-down (about 33%) could be due to any combination with mBest4, a previously reported vesicular mechanism, or other unknown mechanism.

Since previous studies have provided supports for the existence of a volume-sensitive channel as a mediator for exocytosis-independent EAA release, it is likely that mBest1 channels might be regulated by volume changes in astrocytes. In accordance with this idea, each of the following three independently supports the above possibility: 1) a human bestrophin channel (hBest2) is reported to show volume sensitive Cl— permeability, 2) increase in intracellular Ca²⁺ and treatment with hypoosmotic solution can synergistically increase the glutamate release from astrocytes, and 3) a preliminary study by the present inventors of mBest1-expressing HEK293T cells has shown a hypoosmotic solution-induced anion current (FIG. 12). All of these findings suggest that the mBest1 can be activated by both Ca²⁺ increase and volume changes, providing a unified hypothesis about non-vesicular glutamate release which can explain both Ca²⁺ dependence and volume sensitivity of the glutamate release.

The above results establish that mBest1 is expressed in astrocytes and neurons in mouse central nervous system. Also found by the present inventors is a novel function of CAACs in glial-neuronal transmission, suggesting that mBest1 has molecular identity with CAACs in astrocytes. It is demonstrated that astrocytic mBest1 channels can release glutamate by direct permeation. These results suggest that receptor-mediated, Ca²⁺-dependent, non-vesicular and channel-mediated glutamate release from astrocytes have an important role in regulating synaptic activity between neurons. Recently, the bestrophin channel in peripheral neuron was shown to contribute to the amplification of the depolarization by inducing Ca²⁺ activated Cl— efflux. This finding supports the possibility that neuronal bestrophin channel might be widely involved regulating neuronal excitability in the peripheral nervous system.

1. A composition for regulating release of an excitatory neurotransmitter through a Ca2+-activated anion channel (CAAC) from neurons and astrocytes, containing a CAAC activity regulator as an active ingredient.

2. The composition for regulating release of an excitatory neurotransmitter according to claim 1, wherein said CAAC is encoded by Bestrophin 1 gene.

3. The composition for regulating release of an excitatory neurotransmitter according to claim 1, wherein said excitatory neurotransmitter is one or more selected from the group consisting of acetyl choline, aspartic acid, D-serine, glutamate, enkephalin, and histamine.

4. The composition for regulating release of an excitatory neurotransmitter according to claim 1, wherein said CAAC activity regulator is a CAAC inhibitor comprising one or more selected from the group consisting of anion channel blocking agents and antisense RNA or shRNA against a CAAC-coding nucleotide, and has an inhibiting activity against neurotransmitter release through a CAAC from neurons or astrocytes.

5. The composition for regulating release of an excitatory neurotransmitter according to claim 4, wherein said anion channel blocking agent is one or more selected from the group consisting of niflumic acid, flumenamic acid, 5-nitro-2(3-phenylpropylamino)-benzoic acid (NPPB), and 4,4′-diisothiocyanatostilbene-2,2′-disulfonic acid (DIDS).

6. The composition for regulating release of an excitatory neurotransmitter according to claim 4, wherein said antisense RNA is against Bestrophin 1 gene having the sequence of SEQ ID NO: 1 or 2.

7. The composition for regulating release of an excitatory neurotransmitter according to claim 4, wherein said shRNA is one or more selected from the group consisting of sequences of SEQ ID NOs: 3 to 5.

8. The composition for regulating release of an excitatory neurotransmitter according to claim 1, wherein said CAAC activity regulator is a CAAC activator and has activity of promoting release of neurotransmitter through a CAAC from neurons or astrocytes.

9. A composition for preventing or treating diseases caused by over-release of an excitatory neurotransmitter containing one or more selected from the group consisting of a channel blocking agent against a Ca2+-activated anion channel (CAAC) and an antisense RNA or a shRNA against a CAAC-coding nucleotide, as an active ingredient, wherein the diseases caused by over-release of an excitatory neurotransmitter is one or more selected from the group consisting of epileptic seizures, neurotransmitter-induced excitotoxicity, ischemia, brain stroke, brain hemorrhage, epilepsy, traumatic brain injury, and hypoxia.

10. The composition according to claim 9, wherein said channel blocking agent against CAAC is one or more selected from the group consisting of niflumic acid, flumenamic acid, 5-nitro-2(3-phenylpropylamino)-benzoic acid (NPPB), and 4,4′-diisothiocyanatostilbene-2,2′-disulfonic acid (DIDS).

11. The composition according to claim 9, wherein said antisense RNA is against Bestrophin 1 gene having the sequence of SEQ ID NO: 1 or 2.

12. The composition according to claim 9, wherein said shRNA is selected from the group consisting of sequences of SEQ ID NOs: 3 to 5.

13. A composition for improving recognition, cognition, movement, memory, or learning capabilities, said composition comprising a Ca2+-activated anion channel (CAAC) activator as an active ingredient.

14. A preparing method of a channel for an excitatory neurotransmitter release on neurons or astrocytes, comprising the steps of transfecting a mammalian neuron or astrocyte with Bestrophin 1 gene having the sequence of SEQ ID NO: 1 or 2, and expressing the transfected gene.

15. The preparing method according to claim 14, wherein said excitatory neurotransmitter is one or more selected from the group consisting of acetyl choline, aspartic acid, D-serine, glutamate, enkephalin, and histamine.

16. A screening method for a neuroregulatory agent comprising the steps of:
preparing a sample of neuron and/or astrocyte;
contacting said sample with a candidate substance;
testing whether or not a Ca2+-activated anion channel (CAAC) on neuron and/or astrocyte is activated; and
determining said candidate substance as a neurotransmission promoting agent when the CAAC is activated, or determining said candidate substance as a neuroprotective agent when the CAAC is not activated.

17. The screening method according to claim 16, wherein said CAAC is encoded by Bestrophin 1 gene.

18. The screening method according to claim 16, wherein said step of whether or not a CAAC on neuron and/or astrocyte is activated is conducted by measuring inward current change through a sniffer patch method.

19. The composition for regulating release of an excitatory neurotransmitter according to claim 2, wherein said CAAC activity regulator is a CAAC inhibitor comprising one or more selected from the group consisting of anion channel blocking agents and antisense RNA or shRNA against a CAAC-coding nucleotide, and has an inhibiting activity against neurotransmitter release through a CAAC from neurons or astrocytes.

20. The composition for regulating release of an excitatory neurotransmitter according to claim 3, wherein said CAAC activity regulator is a CAAC inhibitor comprising one or more selected from the group consisting of anion channel blocking agents and antisense RNA or shRNA against a CAAC-coding nucleotide, and has an inhibiting activity against neurotransmitter release through a CAAC from neurons or astrocytes.
